(19) United States
(12) Patent Application Publication
(10) Pub. No.: US 2022/0175980 A1
(43) Pub. Date: Jun. 9, 2022

(54) Device and method to inactivate viruses and kill living bacteria

(71) Applicant: Tarek A. Z. Farag, WESTMONT, IL (US)

(72) Inventor: Tarek A. Z. Farag, WESTMONT, IL (US)

(21) Appl. No.: 17/116,394

(22) Filed: Dec. 9, 2020

(51) Int. Cl. A61L 2/00; B01D 46/10; B01D 46/00

(52) U.S. Cl. CPC A61L 2/0047; A61L 2/0023; B01D 2257/91; B01D 46/10; B01D 46/0028; A61L 2/0017

(57) ABSTRACT

A device and process to inactivate viruses and kill living bacteria using the available techniques like UV irradiation, heat, filtration, and/or chemical treatment, individually or all at once to achieve the best results. The operation of the invention could be programmed/controlled using a remote controller/programmer. The invention could be used in spaces occupied by humans or when humans are not present. When humans are not present it uses mainly UV irradiation, and when humans are present it circulates the air to be disinfected through a protective enclosure. In some applications using UV disinfection, the device can circulate and disinfect the air inside it, while irradiating the UV irradiations to other parts of the space to disinfect them. One embodiment of the device is similar to a mask, not just to collect the viruses and bacteria, but also, to inactivate the viruses and kill the bacteria. The invention will make indoor places very safe and safer than outdoors, because the indoor could be disinfected as a controlled enclosed space, while the outdoor is practically impossible to disinfect. Which will allow people to live better life without fears of contracting or spreading the viruses now or in the future. The invention is easy to install, operate, maintain, clean, and highly effective.

Notes:

For the Bacteria to be Killed and the Viruses to be inactivated, we use the term KBV.
The term “disinfection” is used for killing bacteria and inactivating viruses.

### BACKGROUND OF THE INVENTION

### Field of Invention

This invention relates to a method and an apparatus for the use of Ultra Violet radiation (UV), heat, filtration, and chemicals, for killing living bacteria and inactivating viruses (KBV), and in some applications providing illuminations and air purification and freshening. It circulates the air from the surrounding space to the inside of a protected enclosure, where that air is subjected to the proper treatment with one or more of the methods that guarantees high degree of disinfection. And in some cases the device could be used to disinfect the entire place with or without air circulation. This invention allows people to do their normal activities and enjoy their lives without fears of contracting or spreading diseases. This invention could be used in almost any places like restaurants, homes, schools, nursing homes, airplanes, ships, theaters, stores, hospitals, churches, etc.

### Prior Art

The use of Ultra Violet radiation (UV) [Ultra Violet Germicidal Irradiation (UVGI)], chemicals, and heat, for KBV were known for many years. Since UV is hazardous to humans, its use was limited to special situations where humans are protected from its exposure like directing it to the ceiling, inside air ducts, or when humans are not present. One of the references on UV is the report IES CR-2-20-V1 prepared Apr. 15, 2020, by the Illuminating Engineering Society (IES) (www.ies.org). UV lamps are used in air ducts, but their air change rate is dissatisfactory limiting their effectiveness (U.S. Pat. No. 9,157,642). There are many patents that rely on capturing the viruses in filters (U.S. Pat. No. 6,338,340), and others irradiating them with UV after capturing the viruses and bacteria (U.S. Pat. Nos. 5,330,722, 5,817,276, 5,837,207, 6,557,356). Whole-room disinfection using UV from suspended fixtures directing it downward is the safest, most effective application of UV; however, it requires strict precautions. Many small or hand-held UV disinfectants were made, but their effectiveness is small. One application of this invention is to disinfect the air going to a target (person) to protect him, or the air coming out from a source (infected person) to prevent the spread of the viruses, which reduces the amount of circulated air, increases the disinfection exposure (slower flow), increases the effectiveness of the disinfection, and reduces the power demand. An excellent application of the invention is to use it to replace the regular mask to inactivate the viruses to more than 90%, instead of trapping and accumulating them in the mask to become a dangerous source of infection instead of being a method of protection. In some situations, the invention could provide illumination and air freshening effect.

### OBJECTS AND ADVANTAGES

Accordingly, several objects and advantages of my invention are:

(a) to provide a simple and inexpensive method and device for KBV (kill Bacteria and inactivate Viruses) during the presence or absence of people;
(b) to provide a method and device for KBV, which have the advantages as mentioned in (a), and could be used by individuals;
(c) to provide a method and device for KBV, which will have the advantages as mentioned in (a) or (b) and could be used for the entire air volume and surfaces;
(d) to provide a method and device for KBV, which will have the advantages as mentioned in (a) to (c), and require low energy consumption;
(e) to provide a method and device for KBV, which will have the advantages as mentioned in any or all of (a) to (d), and generate light;
(f) to provide a method and device for KBV, which will have the advantages as mentioned in any or all of (a) to (d), and filters and freshens the air; Further objects and advantages are to provide a method and device for KBV, which could be easily used, installed, maintained, moved, adjusted, and controlled. Still further objects and advantages will become apparent from a consideration of the ensuing description and drawings.

### SUMMARY

The present invention is a method and device for KBV (kill living bacteria and inactivate viruses) using all the available techniques, either individually or in variety of combinations (UV, chemical disinfectants, heat and infrared, etc.) [FIG. 2]. The air carrying viruses and bacteria is circulated inside the device and treated safely with one or more of the methods explained. For example; passing the air through a filter containing a disinfectant and irradiating it with UV. Or the enclosure could have controlled openings ((9) FIG. 2, 6, 14), to open them to direct& the UV radiations to all the space and surfaces to disinfect them, when it is safe to do so (e.g. no humans present). Generally, the device could be attached to existing light fixtures to make the installation easy and benefit from the electricity already supplied to the fixture. When the device is using UV, the UV radiations could be directed to the upper part of the room while people are present, with taking precautions that the level of reflected radiations is within the safe limits. Also, visible light (illumination) could be generated from UV (as in florescent lamps and CFL), and in many applications it could be used to replace existing light fixtures. An excellent application is to use the invention to replace the regular mask to KBV, instead of trapping and accumulating them in the mask and eventually the mask becomes a dangerous source of infection instead of being a method of protection.

### DRAWINGS—FIGURES

FIG. 1 shows a cross sectional view of a simplified device of the invention.

FIG. 2 shows a schematic diagram of the general construction of the invention.

FIG. 3 shows a cross sectional view of a simplified device of the invention positioned vertically and using natural convection flow.

FIG. 4 shows a portable device of the invention with adjustment to height and angle.

FIG. 5 shows the layers blocking the UV beam and converting it to visible light.

FIG. 6 shows the strips used to control the openings in the full open-position to allow the irradiation of the entire space when it is safe to do so.

FIG. 7 shows a portable device of the invention that can work as a table-lamp with adjustable height with a flow fan.

FIG. 8 shows the invented device suspended from the ceiling with adjustable height and other remotely controlled features, and a variation of the device of FIG. 7 on the table.

FIG. 9 shows the symbols used for UV radiations, visible light, and air flow.

FIG. 9a Shows the construction of hairy filter to capture bacteria and viruses.

FIG. 10 shows a person wearing the device that does not cover the mouth with inlet and outlet at different sides.

FIG. 11 shows some variations of the invention replacing the regular mask.

FIG. 12 Shows a simplified flow control valve to direct the air flow (in, out, or bidirectional).

FIG. 13 shows a simplified circular disinfecting unit used for masks using UV LEDs.

FIG. 14 Shows a representation of the invention where UV radiations could be directed to the upper part of the space while people are present, and when they are not present to the lower and the upper part at the same time.

FIG. 15 shows a simplified disinfecting unit used for masks using UV LEDs and possibly filtration, chemical treatment, heat disinfection, and de-ionizing member.

FIG. 16 shows a block diagram of the controller used to control/program the device.

### (NOTE: ALL THE DRAWING FIGURES ARE FOR DEMONSTRATION ONLY AND NOT TO SCALE). REFERENCE NUMERALS

| | | | |
|---|---|---|---|
| 1 | UV source | 2 | Air inlet |
| 3 | Air outlet | 4 | Body (enclosure) of the device |
| 5 | Circulating fan | 6 | The device for masks air-in |
| 7 | The device for masks | 8 | The device for masks air |
| | air-out | | in/out |
| 9 | Open/Close strips | 10 | UV Transparent material |
| 11 | Layer to convert UV to | 12 | Light transparent/UV blocker |
| | light | | layer |
| 13 | Air direction valve | 14 | Filter member |
| 15 | De-ionizer | 16 | Heating disinfectant |
| 17 | Filter for chemical | 18 | Disinfectant dispenser |
| | disinfectant | | |
| 19 | Captured bacteria and | 20 | Hairs to capture bacteria and |
| | viruses | | viruses |

### DRAWINGS REFERENCE LETTERS, ABBREVIATIONS, AND SYMBOLS

C Central-programmable/controller unit.
F Fan control.
H Humidity sensor
O Opening control for inlets or outlets.
P Position control (up/down/angle).
R Remote controller/programmer.
S Sensor (motion or presence of humans).
T Temperature sensor.
U UV source power/control.
cm2 centimeter square (cm×cm), m2 meter square (m×m).
cm3 cubic centimeter (cm×cm×cm), m3 cubic meter (m×m×m).

### DETAILED DESCRIPTION

One of the main ideas about the invention is that the prior art focuses on the whole volume from a distance, while this invention works very close to the area of interest. For example, in a room of 5×5×2 m of volume 50 m3, instead of disinfecting the entire room starting at the ceiling, we can start the disinfection close to the persons we need to protect, which could be a volume of less than 1 m3 around them, reducing the disinfection demand by 1/50, and on the same time disinfect the whole room starting from this point.

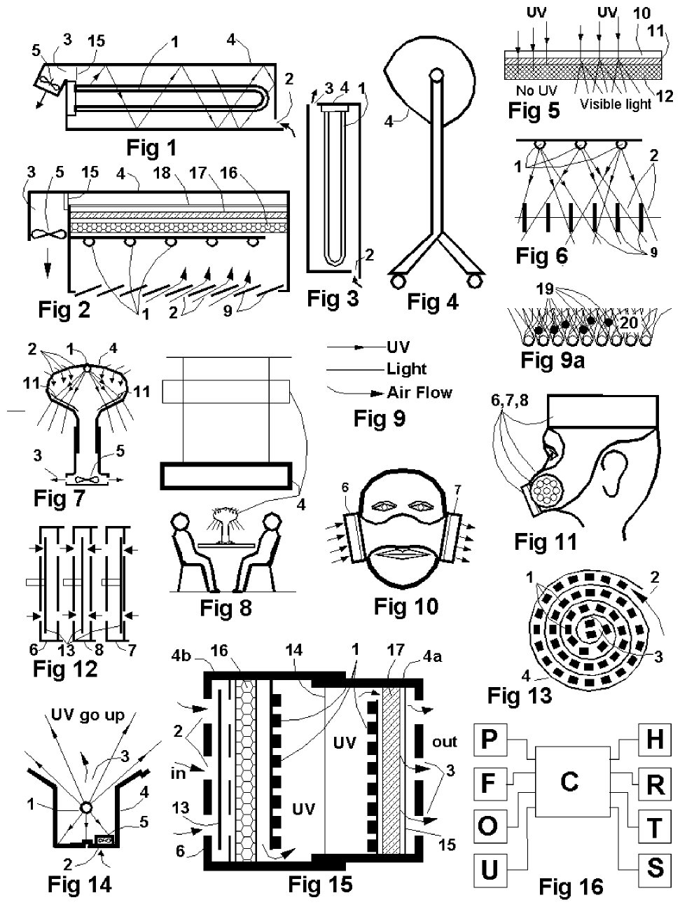

The device of FIG. 1 shows a simplified construction of one of the embodiments of the invention using UV. It has an UV source (1) [shown as a single lamp, but could be an LED array, pulsed xenon arc lamps, krypton-chlorine excimer lamps, etc.] irradiating inside an enclosure (4). The enclosure (4) is provided with an air inlet (2) and air outlet (3), both can take any form, shown here as simple openings. The air could be driven through these openings with fans (5), or external forces like external ceiling fans. The inside of the enclosure (4) is preferred to have good UV reflective characteristics (shown in FIG. 1), to increase the effectiveness of the radiations and reduce its absorption and heat generation. The air carrying viruses and bacteria enters the enclosure (4) through opening (2); where it is subjected to UV from source (1) and its reflections, with amounts of radiations enough to disinfect the air. The reference IES CR-2-20-V1 (page 14/24), estimates the radiation dose for surfaces from 20 to 100 mJ/cm2 [depends on the type of surface and its cleanliness] to disinfect surfaces to 99%; and about 17 mW/m3 for volumes, which could be achieved easily with UV LEDs. There are UVC LEDs' Lamps containing 130 LEDs of 40-Watt power for $10, i.e. 307 mw/LED.

Another embodiment is shown in FIG. 2, where the UV source (1) is a plurality of sources; and an inlet (2) and outlet (3). Both can take any form; like simple openings or a plurality of controlled strips (9) that could be adjusted to control the flow and prevent UV from going outside. Those strips (9) could be fully open to allow UV to irradiate the outside space when humans are not present as shown in FIG. 6. Further, strips (9) could be used to generate light by converting UV into visible light. Strips (9) could be made from a layer (12) of visible-light transparent material and UV blocker, covered with a layer (11) (fluorescent) to convert UV into visible light, and covered with protective layer (10) transparent to UV. The unit could have a heating element (16) that could be made from resistive strips or spongy ceramic heater to trap the viruses and bacteria. Passing electric current in the heating element (16) will increase its temperature for disinfection. It could be made hot continuously or pulsed to a temperature enough to do the disinfection [if pulsed, the electric current is switched on for short period of time (0.5 to 3 seconds), and then off for longer period to cool down (1 to 10 minute)]. The heating element (16) could be made of an array of electrical heaters to be activated in sequence individually to reduce the current surge or all at once. The unit could have a chemical disinfectant member (17) that could be made to trap the viruses and kill them chemically (e.g. woven fabric with hairy (fibers) covering (20), wetted with disinfecting gel). The unit could have a disinfectant dispenser (18), to dispense the disinfectant to the chemical disinfectant member (17) [to keep the disinfectant active (not to dry or lose its effectiveness)]. Also, the unit could have a collector for the spent disinfectant (not shown), or the disinfectant member (17) itself could be changed at the end of its effective life. In some cases, the UV could cause undesired ionization, for which the deionizer (15) could be provided to de-ionize the air and in some cases could be used to attract ionized dust purifying the air adding freshening effect. The deionizer (15) could be made from a conductive or resistive mesh or strips, or activated carbon, even the electric heater could be used as a deionizer. It should be noted that two or more of the 4 members (15), (16), (17), and (18), could be integrated, or eliminated.

In some applications, it could be convenient to replace a light fixtures with the invention or attach the invented device to it, as shown in FIG. 8. In case of replacement, the unit could be used as a light source by adding light bulbs to the device or LED light, or by converting the UV to light (UV was used for tens of years to generate light). The surface to emit the light, could be made of a material transparent to light (12) and preferably opaque to UV as shown in FIG. 5, and a layer of material to convert UV to light (fluorescent material) (11) is deposited on it, and above it a layer of protective material (10).

The invention could be made in many forms. For example, FIG. 3 shows an embodiment where the unit (4) is installed vertically. FIG. 4 is a portable unit on a stand where its height, angle, and other functions are controlled or programmed. FIG. 7 is for a unit that resembles table-light that could have adjustments to height, power, flow, etc. FIG. 8 shows the device in an application (like a restaurant) where the unit (4) could be raised to the higher position when people are not present to disinfect the whole place with UV radiations as explained in FIG. 6. When people are present the unit (4) is lowered to its low position to disinfect the air close to the people by sucking the air and circulating it inside the unit to disinfect it by UV radiations and/or other means as explained before. Additionally, a unit (4) similar to FIG. 7 could be placed on the table to disinfect the immediate air around their heads, and provide light as a table-light. FIG. 14 Shows a representation of the invention where UV radiations could be confined to the inside of the unit by closing all the sides to contain the air and disinfect it. Or when conditions allow other modes of disinfection by UV, the suitable side of the device could be opened. For example, we close all the sides to disinfect the coming air while people are present, or open the upper side only to disinfect the coming air and the upper part of the space while people are present, or open the upper and lower sides (or open all the sides) to disinfect the whole space when people are not present. To demonstrate by numbers, let us assume that 8 persons are eating together in a restaurant at one table in an area 4 m2 (2×2) and the height of the ceiling is 3 meters giving 12 m3 volume. Each person will breathe about 0.5 liters of air 15 times per minute, i.e. their total is 60 liters/minute. If using a device with dimensions comparable to a fluorescent lamp of 1.2×1×.25 meters (0.3 m3 volume), it will store 5 minutes of air for their breathing, which is very easy to disinfect using UV source. If using a small fan of 700 liter per minute, it can circulate and disinfect the whole volume of 12 m3 in 17 minutes while people are present.

A device like the one shown in FIG. 2 could be used in air ducts.

The Invented Device for Masks

Regular masks have many disadvantages. They DON'T KILL BACTERIA OR INACTIVATE VIRUSES; their abilities to prevent viruses from passing through them is very small; they obstruct the air going in/out of the lungs requiring extra effort from the person wearing them; they reduce the air flow and rate of exchange of oxygen increasing the levels of carbon dioxide in body; people have the tendency to reuse them without disinfection; they obstruct the social interactions between people; the dangerous part is that they may ACCUMULATE the bacteria and viruses and BECOME A DANGEROUS SOURCES OF INFECTION to the persons wearing them and to others instead of protecting them; and they could give a dangerous false feeling of safety.

FIG. 10 shows two units installed on a mask. Unit (6) is for inhaling to protect the person, and (7) for exhaling to protect others.

The invention in its simplest form could be the device of FIG. 15 with only the filter (14), preferably made from woven fabric [FIG. 9a] with hairy covering (20) wetted with sticky material (like gel), which simulates human's respiratory system in that it allows air to pass easily through it while providing a large area and mechanism to capture (stick) the viruses and bacteria (to the hairs). This filter (14) could work without adding to it a chemical disinfectant. After using the filter (14) for the appropriate time, it could be replaced with a fresh one; or removed, disinfected by any means available (heat, UV irradiations, chemical agent, boiling water, etc.), cleaned, and reactivated then installed back. FIG. 15 is shown in its general form. It has UV source LEDs (1), filter (14), directional valve (13) to make the unit input only, output only, or input/output, filter for chemical treatment (17), heater (16) for disinfection, and de-ionizing member (15).

FIG. 11 shows the embodiment used by a person for KBV of the air coming into or going out of his lungs. The diagram shows units (6), (7), and (8), however, the number of units and configurations could vary according to the needs. FIG. 10 and FIG. 11, demonstrate the power and flexibility of this invention. For example, in the case of a virus infected person, our goal would be to protect others, hence we can use 3 units; two units on the side could be configured for out-flow, and one unit directly on the face for in-flow. FIGS. 10 to 13 and 15 show some details of these units and other variations. The unit on top of the head (like a hat) of the person in FIG. 11, is designed for the cases using UV LEDs as shown in FIG. 13, to store the air to allow time for disinfection. For example, assuming the normal respiration rate for an adult at rest is 12 to 20 breaths per minute (assume average 15 per minute); the average volume of air entering the lungs is 0.5 liters per inhale; and average diameter of the head of an adult 17.5 cm. From the reference: https://www.ncbi.nlm.nih.gov/pmc/articles/PMC7112078/ a dose of 370 J/m3 disinfected 98.5% of the bacteria in 15 seconds, i.e. about 25 W/m3 (compare this to the 17 mW/m3 stated before); then in 1 minute a person will inhale [0.5 L×15 per minute=7.5 liter/min] and in 15 seconds 7.5×(15/60)=1.875 L in 15 sec.]. Then the LED power needed will be [25 W×1.875 L/1000=46.875 mW], which is very easy to meet with few UV LED. Assuming that the mentioned UV LED of power 307 mW, converts input power to UV at low efficiency of 2.5%, i.e. 7.7 mW UV per LED, then we need 6.1 LEDs, then we use 7 or 8. To have the hat of FIG. 11 hold 1.875 liters, its height will be about 8 cm. FIG. 12 shows valve (13) in different configurations; left position for in-direction configuring the unit for inlet (6), middle position for bi-direction in/out making unit (8), and right position out-direction making unit (7).

Although the description of FIG. 15 is focused on the application for a mask, this configuration is also applicable to other applications. The units of FIG. 15 have two-part body (4a) and (4b) to allow opening the unit for cleaning and maintenance. If unit (8) is configured as bidirectional, the amount of irradiation could be controlled differently according to the direction of the flow using the control unit of FIG. 16. FIG. 15 shows the unit configured as (6) with the flow going to the lungs. Valve (13) will move to block the inlet openings when the air tries to move opposite to the arrow. Valve (13) could be configured to allow the air to go out from the lungs making unit (7), or centered to make unit (8). The best UV source (1) for these units is an array of LEDs emitting UV and operated by batteries or low voltage power supply. These units could be provided with a filtering member (14) (preferable made from a polymer transparent to UV) to trap some of the bacteria and viruses (not required to trap them all) to extend their exposure time to UV, improving the effectiveness of the unit. These units could be provided with neutralizing mesh (15). Using UV in units (6), (7), and (8) have huge advantages compared to masks or similar methods. In addition to what mentioned before of killing the bacteria and inactivating the viruses (not just collecting them), they could be reused, easily cleaned, and be self-disinfecting by running the unit without air flow for few seconds (may be holding the breath). They let the air move in and out with very low resistance, without adding loads on the lungs. The functions of the members (14) to (18) are explained before, and any combinations of them could be used, in addition, they could be integrated into one or more members.

The Central-Programmer/Controller Unit

FIG. 16 shows a block diagram of the central-programmer/controller unit (C) used generally for all the devices, and the description here will be with reference to a device having the full features. The temperature monitoring signals (T) to protect the device against temperature rise due to the heating effects of UV and other radiations or the heater element if used. A safety signal (S) indicating if there is a human in the area. (S) could be generated by different methods like motion sensing or IR sensing. A humidity sensor (H) is used to measure humidity and indicate if the disinfectant chemical is dry or there is water condensation. A remote controller (R) is used to communicate with (C) to control and program it. The controller (C) will control the positioning (P) for the height and the angle of the unit; (O) for opening and closing strips (9) to control the air flow and the full irradiation of the entire space; (F) for the fan (5) speed and direction of rotation (flow); and (U) for the UV source (1) for intensity of the irradiation and pulse rate if pulsing source is used. As an example of programming, let us assume a classroom that we need to protect it from viruses using the invented device. Assume the class will start at 7:00 am and ends at 4:00 pm. We can program the device to be at its high position (P) to disinfect the entire classroom and its surfaces [send signal (O) to fully open strips (9), and send signal (F) to activate UV source (1)] for 30 minutes starting at 6:00 am until 6:30 am; then send signal (P) to lower the device at 6:45 am and close the strips (9); at 7:00 am run the fan (5) and keep (U) ON until 4:30 pm; lift the unit up at 4:30 pm and open strips (9) to start irradiation cycle for 30 minutes; then turn the unit OFF until 6:00 am next day, to start a new cycle. Notice that the programmer can take care of holidays, weekends, power interruptions, etc.

Thus the scope of the invention should be determined by the appended claims and their legal equivalents, rather than by the examples given.

I claim:

1-20. (canceled)

21. An apparatus for disinfection (killing bacteria and inactivating viruses) comprising:
a. an enclosure means;
b. at least one means to kill bacteria and inactivate viruses;
c. an air inlet means to said enclosure means; and
d. an air outlet means to said enclosure means.

22. The apparatus for disinfection of claim 21, wherein said disinfection means is a filtration means.

23. The apparatus for disinfection of claim 21, wherein said disinfection means is a source of UV irradiation means.

24. The apparatus for disinfection of claim 21, wherein said disinfection means is a heating means.

25. The apparatus for disinfection of claim 21, further comprising: a central-programmer/controller means; temperature signal means; humidity signal means; safety signal means; and a remote programmer/controller means, and further comprising in/out/in-out air flow control means; position control means; opening/closing control means; fan speed and direction control means; wherein said remote programmer/controller means will program and control said central-programmer/controller means to control the disinfections.

26. The apparatus for disinfection of claim 23, further comprising visible light (illumination) generation means.

27. The apparatus for disinfection of claim 25, wherein the sides of said enclosure means are provided with controllable mechanism means to open or close said sides, wherein said central-programmer/controller means controls said controllable/mechanism means such that to open or close the sides of the enclosure in a way to allow said UV irradiation to irradiate the spaces inside and outside said enclosure means.

28. The apparatus for disinfection of claim 22, wherein said filtration means is a woven fabric with hairy coverings provided with sticky material means to trap bacteria and viruses; and further comprising a chemical treatment means to disinfect said filtration means, and a suitable applying means to apply said chemical treatment means.

29. The apparatus for disinfection of claim 21, suitably designed to use at least one variety of the apparatus by a single person.

30. A process for disinfection comprising the steps of:
a. preparing an enclosure suitable for disinfection;
b. providing said enclosure with at least one suitable disinfection process means;
c. providing an air inlet process means to said enclosure that needs disinfection;
d. providing an air outlet process means to said enclosure to exit the air after disinfection;
e. introducing the air to disinfect into said enclosure using said air inlet process means;
f. acting said disinfection process means on the air introduced into said enclosure; and
g. exiting the air that was disinfected using said air outlet process means.

31. The process for disinfection of claim 30, wherein said disinfection process means is an UV irradiation process means.

32. The process for disinfection of claim 30, wherein said disinfection process means is a chemical process means.

33. The process for disinfection of claim 30, wherein said disinfection process means is a filtration process means comprising suitable chemical agent process means.

34. The process for disinfection of claim 30, wherein said disinfection process means is a heating process means.

35. The process for disinfection of claim 31, further comprising additional process means to irradiate the space outside of the enclosure means when it is safe to do so.

36. The process for disinfection of claim 30, further comprising additional process means to flexibly reconfigure the process of inlet to a process of outlet or inlet/outlet, or the process of outlet to become a process of inlet or inlet-outlet.

37. The process for disinfection of claim 30, further providing: a temperature signal process means; a humidity signal process means; a safety signal process means; a remote-programming/controlling process means, and a central programming/controlling process means; and further comprising an in/in-out/out air flow process means; a position controlling process means; an opening controlling process means; a fan speed and direction controlling process means; and a process means to control said disinfection process.

38. The process for disinfection of claim 30, further providing a process means to generate illumination.

39. The apparatus for disinfection of claim 21, wherein said enclosure means is having enough capacity to hold air to be disinfected that would allow more time for disinfection.

40. The apparatus for disinfection of claim 21, further comprising a portability means to move it wherever needed, height adjustment means, and tilt adjusting means, wherein said enclosure means is constructed from openable/closable sections to allow flexibility for air to get out or in said enclosure, flexibility in disinfection, and ease of maintenance and cleaning.
